(19) United States
(12) Patent Application Publication
(10) Pub. No.: US 2011/0105856 A1
(43) Pub. Date: May 5, 2011

(54) Diagnostic testing

(76) Inventors: Robyn Aylor Haines, Columbus, OH (US); Robert Benson Aylor, Cincinnati, OH (US)

(21) Appl. No.: 12/925,221

(22) Filed: Oct. 13, 2010

Related U.S. Application Data
(60) Provisional application No. 61/280,146, filed on Oct. 29, 2009.

(51) Int. Cl. A61B 5/00

(52) U.S. Cl. 600/300

(57) ABSTRACT

One or more living entities are evaluated for health conditions by taking samples, especially samples of volatile compounds, derived from, e.g., the breath, urine, feces, saliva, skin exudates, etc., from the living entities, optionally with concentration of the samples; analyzing the samples using a multivariate analytical test; comparing the patterns present in the results of said test or tests, either; a. on an individual entity at a previous time to the patterns present in the results of said test or tests on the individual entity at the present time, or b/ comparing the patterns present in the results of the test on the said living entities to the patterns present in the results of tests on populations of similar entities having a normal health condition and/or to the patterns in the results of tests on populations of similar entities having a given disease or abnormal health condition, the results from the entities having normal health and each of the populations of entities having a given disease or abnormal condition being treated as reference sets for their respective specific disease or abnormal condition as compared to the reference set for entities having normal health.

This application claims priority from U.S. provisional application Ser. No. 61/280,146, confirmation No. 2290, filed Oct. 29, 2009, said provisional application having the same title and inventors. Said provisional application is incorporated herein by reference.

### BACKGROUND OF THE INVENTION

1. Field of the Invention

The invention relates to methods for determining the condition of living entities, especially people and animals, by analyzing the materials, and especially volatile materials, given off by such entities and desirably combining these methods with other diagnostic techniques to improve the diagnosis of the condition of the living entities. As used herein, “entities” is used to refer to living organisms, especially humans and animals. “Animals” especially comprises pets, food animals, laboratory animals, etc.

2. Brief Description of Related Art

Animals and humans give off many volatile materials, e.g., in their breath, from their skin, from their urine, and from their feces on a regular basis and at other times from, e.g. wounds. There is a considerable body of knowledge suggesting that the odors given off by living entities can identify the existence of various illnesses like measles, renal failure in cats, etc. Animals, especially dogs, and even humans, can detect odors that are associated with various conditions and illnesses, and thereby diagnose the presence of such conditions and illnesses. There have been many observations and some studies of the ability of animals such as dogs to determine the threat of heart attacks, strokes, epileptic seizures, and/or the presence of cancer.

The subsequent scientific attempts to mimic animal noses use simple analytical techniques and/or try to use specific “markers” believed to be indicative of certain illnesses. One approach uses the “artificial nose” which makes use of a collection of receptors that are supposed to mimic the human nose. The artificial nose makes use of a variety of receptors to discriminate between different chemical classes of compounds, but does not provide enough information to distinguish between minor variations in compounds. Since these approaches are not very sensitive, many studies involve how to concentrate the markers for more reliable predictions. Examples of such work are disclosed in U.S. Pat. Nos. 5,465,728, issued Nov. 14, 1995; 5,848,975, issued Dec. 15, 1998; 5,996,586, issued Dec. 7, 1999; 6,221,026, issued Apr. 24, 2001; 6,245,547, issued Jul. 3, 2001; 6,312,390, issued Nov. 6, 2001; and 6,540,691, issued Apr. 1, 2003, all to Phillips. There is also a published Patent Application US 2003/0109794 A1, published Jun. 12, 2003 by Phillips that relates to methods of improving such methods by concentrating the sample. The methods relate to detecting the amounts of only a specific few of the many volatiles present in breath which have been identified as “markers” for certain diseases.

The art appears to be primarily directed toward confirming the existence of specific diseases by the presence of certain levels of particular “markers”.

### SUMMARY OF THE INVENTION

The invention comprises methods for determining the condition of a living entity using at least one multivariate analytical test method wherein materials, especially complex volatile materials, given off from said living entity are analyzed with said test method, or methods, to provide test results, and those results are then compared to one or more reference sets based upon the results from the same kinds of test methods on either the same entity at a time when the entity's condition is known, or on one or more reference populations of similar entities, where each of the reference populations has a known condition. The existence of a difference, or of no difference, between the results from a test on an individual and at least one reference set, can establish, e.g., whether there has been a change in the condition of the individual and/or whether the individual shares one or more of the conditions of the individuals comprising the reference populations of the reference sets that are closest to the results obtained from the test individual.

The method is especially useful as an early step in determining whether an individual is ill and what is the most likely cause of the illness. Thus, the methods disclosed herein are especially useful as diagnostic tools that can help medical professionals evaluate the health of, e.g., humans and/or animals. The method is especially useful as a first screening test to suggest subsequent steps in the diagnostic effort. The method speeds the final diagnosis, thus helping to minimize damage caused by the ongoing progression of, e.g., disease.

Thus, the invention herein can comprise one or more of the following:

(1) the step of developing one or more data bases which comprise one or more reference sets (collection of data derived from tests on a population of individuals having a shared condition), created by analyzing complex materials given off by different populations of living things, each population sharing a known condition such as “good or normal health”, specific diseases, “abnormal conditions” such as diabetes, inherited abnormal conditions, etc.;

(2) the step of using data mining software to establish patterns based on the results of tests on populations sharing a known condition to use as “markers” for those different known conditions (The “markers” in this procedure are patterns rather than a specific minimum level of a specific compound. Combining the results from all of the living things having the same condition provides the basis for identifying “patterns” that are associated with given conditions.);

(3) the step in which the results from an individual can be compared to all or some of the said reference sets at one time (Thus, the methods are desirable for helping in diagnosing illnesses based upon comparison of the results from an individual to the reference sets representing different diseases.);

(4) the step of comparing the results from an individual to reference sets derived from one or more healthy populations having similar genetic backgrounds, allergies, family histories of susceptibility to diseases, diets, etc., to determine if the individual shares the same inherited basic characteristics as one of the populations;

(5) the step of using one of the methods herein to provide a shortcut to the diagnosis of diseases;

(6) the step of using patterns found by one of the methods herein to determine what body processes are related to different diseases;

(7) the step of using one of the methods herein to monitor the progress of a condition, e.g., during treatment either directly or by identifying specific markers that vary proportionally with the progress of a specific condition;

(8) the step of using one of the methods herein to determine when changes occur in a simple entity, e.g., a microorganism or virus;

(9) the step of providing the data found using one of the methods herein and/or the methods used to practice said method(s) in digital form for storage, retrieval, and/or analysis, e.g., by at least one computer;

(10) the step in which one or more volatile materials are collected, optionally repetitively and desirably in a standard way, from at least one individual living entity and analyzed using said multivariate analytical test method; and

(11) the step of creating a reference set by analyzing volatile materials from several entities that are similar to each other in at least one relevant aspect and combining the data into a “reference set” to provide a basis for comparison to the results from an individual (Although the reference set can be treated as a “point” there will be variance within the results comprising any set, so it is desirable to treat the results for any condition as a set.).

The invention can also comprise one or more of the following:

(1) a step of using a computer and “pattern recognition” software for said computer to determine if one or more “reference set” patterns exists within the results from a multivariate analytical test method or methods on either an individual, or several individuals sharing a similar condition, when compared with the results from one or more existing reference set patterns derived by similar testing of an individual or several individuals having a known condition;

(2) a controlled diet for one or more individuals so that when they are tested by one or more multivariate analytical techniques as discussed herein, the results more clearly reflect the condition of the individuals tested by minimizing variables affected by diets, especially when the condition is one, e.g., diabetes, that is affected by the diet;

(3) a health care system in which data from said multivariate analytical methods are collected at one or more times from one or more individuals and kept in a data bank, optionally a central data bank, for later comparison with new results either before, or after, characterization using pattern recognition software;

(4) a step of providing digitized representative pictures representing the appearance of individuals so that one, or more, of the pictures can be selected as representative of an entity for purpose of including in the data used for diagnosis;

(5) a step of adding to a digital data base for an individual the temperature, blood pressure, etc., taken at one or more times, optionally said data being taken from one or more monitors that take the data automatically;

(6) a health care system in which a health care professional incorporates one or more of variations (1)-(5) into their practice;

(7) a process in which one or more of the basic invention concept or variations (1)-(5) are part of a method of determining the health of animals, including, optionally, either pets or animals intended for use as food;

(8) a process comprising analyzing the results from several tests of individuals having a common condition using multivariate analytical test methods to determine sub sets of variables that are most closely related to a given condition;

(9) the graphical representation, e.g., by spider diagrams, of patterns and/or analyses of unknown samples of the preceding variations for purposes of understanding and/or comparison (This is especially helpful when the users of the methods have different native tongues and/or lack of computer access.);

(10) a method comprising collection of samples from a specific entity and initial analysis by the multivariate analytical method or methods described herein at a point near the specific entity, followed by pattern analysis and/or evaluation at another, e.g., more central, location;

(11) a method of improving the health of individuals by providing feedback on positive and/or negative results obtained from changes in diet, medicines, behavior, etc. using the processes herein;

(12) a method of determining the effectiveness of medicine, diet, behavior changes, etc., by monitoring the effects using one of the methods herein;

(13) a method for monitoring the health of a single individual by comparing new results to historical results, especially results from when the single individual was “normal”;

(14) a method for providing a basis for insurance risks utilizing one of the methods herein;

(15) the step in which one or more of the database(s), and especially the reference set(s), are copyrighted to provide a legal basis for protecting them from theft, and/or to provide a basis for receiving compensation for the use of the databases and/or reference sets to support the maintenance and improvement of such databases and/or reference sets; and

(16) the step of providing standard food diets for test subjects to minimize differences resulting from foods (This is very desirable for animals, children, etc., where the diet can be readily controlled and where it is difficult to obtain input from the test subject.).

### DETAILED DESCRIPTION OF THE INVENTION

Although it is known that the volatiles (like odors) associated with humans and animals, e.g., from the breath, urine, etc., can be used to diagnose certain diseases, the current state of knowledge uses tests either for markers that are specific to one disease, agent, etc. or uses noses of canines or humans. Human noses are not as good as canine noses and both require talented individuals. The use of methods, such as those described herein can be used to screen for many different diseases and agents simultaneously and/or speed the identification of “markers” for different diseases and the development of more specific tests for target populations. The tests herein, with appropriate reference sets, can be used for diagnostic purposes to provide health care professionals and public safety officials with fact based information that identifies the most likely condition of whoever or whatever is being tested.

It is difficult to establish which specific volatile materials are the “markers” that are related to specific abnormal conditions without considerable effort. Moreover, many “markers” can indicate more than one condition. The generation of more information on many volatile materials provides patterns which can be compared, desirably using a computer with pattern recognition software, to the same type of information derived from the same entity, related entities, similar entities, or, desirably, one or more populations of similar entities having known conditions. Similar patterns will suggest that the condition of the test entity is the same, or similar to, or different from, the condition of the entities having the known condition(s).

Applicants use multivariate analytical techniques to quickly diagnose many different changes in living entities by first analyzing the volatile materials from, or other samples taken from, standard entities or organisms to create reference sets comprising a large amount of data and then obtain similar data from test entities and compare the test entity data to the reference sets. This large amount of data would normally overwhelm the health care provider or researcher. However, by combining pattern recognition software with the multivariate analytical techniques and utilizing complex reference sets derived from the same types of living entities, it is possible to obtain a preliminary diagnosis immediately based upon the similarity of patterns. The method works best when the data is in digital form so that computers can be used to compare and analyze the data.

If one creates a series of “reference sets” that represent known populations representative of both normal, and abnormal, e.g., sick, populations where the cause of the abnormality is known, for specific classes of entities, a computer with appropriate “pattern recognition” software can be used to compare the results from an individual with the known reference sets and determine which reference set or sets best “fit” the results.

Thus, generically, the invention comprises a method for determining the condition of a living entity comprising at least one step selected from:

a. creating one or more reference sets using at least one of the methods utilizing at least one multivariate analytical method as described herein, desirably by combining the results from the same, or similar, living entities having similar conditions to create said reference sets;

b. analyzing at least one sample of volatile material, similar to the materials used to create the reference set(s), from at least one specific living entity using the same multivariate analytical method(s) to provide a specific result representative of the condition of said specific living entity at a specific time; and

c. comparing the specific result of b. with one or more of the reference sets of a to determine which of the reference set or sets is closest to said specific result.

This method is highly desirable for use in a clinical evaluation of an individual entity as discussed before, but is extremely desirable for screening a large number of individual entities to determine if a problem exists, or using appropriate software and additional information about the individuals, determining if a common variable is responsible for a condition, what volatile materials are associated with a condition, etc.

Therefore, a desirable aspect of this invention is the combination of a vapor phase mass spectrometer and “pattern recognition” or other “comparison” software to compare the results of vapor phase spectrometry testing of volatiles from a specific entity, or group of entities, with a set of reference sets, desirably comprising at least one standard representative of a population of healthy entities like the specific entity(s).

This method is especially useful when the living entity is a mammal, and especially when the samples are obtained from the breath, urine, feces, or skin of the mammal and/or similar mammals, and the comparison of the results is accomplished using the digital expression of the results, a computer, and pattern recognition software.

An advantage of the methods herein is that they can be relatively non-invasive. Another advantage is that the analytical devices and computer equipment can be used for their normal uses when they are not being used in the methods herein since they are not limited to specific methods.

A. Multivariate Test Methods

Thus, in a broader aspect, the present invention relates to using one or more multivariate analytical techniques to analyze, e.g., volatile materials from around, and/or samples removed from, specific entity (the “unknown”) to give a series of results (values) that can be compared with the results obtained from making the same analyses on one or more populations of similar entities having similar conditions to create one or more “reference sets”. The comparisons are made using “pattern recognition” software to find the reference set that is most similar to the results found for the specific entity. This method allows one to quickly establish with a relatively high degree of accuracy the health and/or status of the specific entity (living organism). The pattern of results is typically more revealing than the results from analyzing for the presence and/or amount of any one specific volatile compound.

The term “multivariate analytical test method” typically refers to test methods capable of determining continuous variation in the components of mixtures of materials to desirably reflect variations like isomers, homologous compounds, etc. and specifically excludes the “artificial nose” methods which do not provide sufficient information and which are affected by the presence of materials that have no relevance. As used herein, “multivariate (or multivariable) analytical test” refers to analytical techniques that provide results for many different components of a mixture, e.g., typically more than about 30, especially more than about 50, variables, in a single analytical procedure, especially vapor phase mass spectrometry, although for purposes of maintaining a central data base or incorporating results from tests into a health care program, one can include less complete analytical tests for the limited data that they can provide. Other multivariate analytical test methods include nuclear magnetic resonance spectroscopy, Fourier transform infrared spectroscopy, Raman spectroscopy, etc., when the samples from the entity are susceptible to such analyses. Nuclear magnetic resonance spectrometry results provide additional data that can be very useful in detecting differences in very similar materials.

Some of these methods allow for examining non-volatile materials, desirably those that do not require invasive collection means. The use of multiple methods can provide more information, although increasing the amount of data that needs to be processed.

The samples are desirably volatile and the analyses of the samples are desirably obtained using a vapor phase mass spectrometer but the complex material can be analyzed using headspace mass spectrometry, and/or nuclear magnetic resonance spectroscopy, and/or Fourier transform infrared spectroscopy, and/or Raman spectroscopy. As described above, the results of such analyses are desirably then analyzed by a pattern recognition program to determine the degree of similarity to one or more reference sets and the results are desirably given in digital form. Digital form is especially desirable for sharing the results, especially in epidemiological evaluations. In typical health care methods herein, the result of a test on a specific entity is compared to at least one complex reference set representative of either the history of the entity itself or appropriate populations of similar entities under similar conditions, where the condition or conditions of such populations are potentially similar to the condition of the specific entity.

A highly desirable analytical method uses a rapid gas (headspace) analyzer that combines mass spectrometry and chemometrics for discrimination and classification of volatile material samples. This chemical sensor typically relies on a scan of mass fragments over an m/z range of 35-200 of the total headspace, which essentially provides 165 nearly independent channels of information. Chemometric software is typically then used to organize that information and build models for discrimination and classification.

Vapor phase mass spectrometry, an analytical technique that is very useful in the practice of the invention, can be used in the methods herein for determining normal and/or abnormal conditions in living entities, especially mammals, which give off volatile materials as part of their existence. Vapor phase mass spectrometry can provide results very quickly, and can use samples taken by non-invasive means rather than, e.g., from blood taken by a needle. Speed is important in providing health care, thus using the method in association with a central database and internet connections can efficiently provide information anywhere in the world that is connected to the internet and can supplement the necessarily limited facilities available at the initial health care provider's location.

A specific gas, e.g., headspace, analyzer that is especially useful in the present invention is a “chemical sensor” which combines a mass spectrometer that is adapted to sample the headspace with software that provides a pattern recognition capability to provide data analysis and classification. An instrument that is well suited for use in the present invention is the Gerstel ChemSensor 4440 Chemical Sensor system. The Gerstel ChemSensor 4440 Chemical sensor system has four components: an HP 7694 headspace automatic sampler for introduction of gas/headspace samples, a mass spectrometer with a sensor array using quadrupole technology, an integrated chemometric/pattern recognition software package from Infometrix, Inc., for instrument control, data analysis, and reporting, that is loaded in a high-performance personal computer. The whole mixture of gas/headspace volatiles from a sample is transferred directly to the mass spectrometer where all of the volatiles are fragmented and ionized. The mass spectrometer is designed to register the signals of the resulting charged ions over a user-selectable range from 2 to 800 atomic mass units. This results in a pattern that is unique to the particular sample. Pattern recognition and clustering software are capable of differentiating and grouping samples according to their unique mass spectral pattern or “fingerprint”. Samples that are more similar to one another in volatile chemical composition, and which are representative of the status of the entity from which they are obtained, are grouped more closely together, while samples that differ widely will be far apart.

The tests desirably provide information relating to the identity and/or concentration of most, or all, of the components of the volatile materials. The combination of multivariate tests and pattern recognition software provides distinctive patterns for individuals that can be associated with and/or compared with similar patterns from the same individual(s) at different times and/or different individuals and/or groups of individuals. Current methods for the investigation of diseases which use analyses of saliva, blood, etc. to determine the existence of a disease can be improved by using patterns as well as individual “markers” to find and/or monitor health conditions. Patterns are more reliable and easier to detect than absolute levels of individual components typically used as “markers”.

B. Sample Collection

Depending on the entity or organism, samples of volatile materials given off by the entity are obtained either from the atmosphere over an entity or a population of the entities, the breath (respired material), one or more waste products given off by the entity, treatment of a solid sample (skin scrapings, biopsy, etc.) taken from the entity, etc.

Volatile materials are created as a result of natural processes in living entities, so the results of multivariate analytical tests on individual samples or mixtures of samples of such volatile materials, e.g., from urine, feces, breath, saliva, skin, etc., collected from a living entity can provide useful information relative to the health of that living entity. The results of, e.g., the vapor phase mass spectrometry tests, provide information about the amounts and relative amounts of the volatile organic materials created by an entity which then can be used to assist in the evaluation of the health of the entity. Such tests can also provide early warning of mutations in, e.g., diseases, when the pattern changes.

The volatile material can be used as collected, or can be optionally, but desirably, concentrated. The sample can be collected, either concentrated, or collected and then concentrated, by one or more techniques such as putting the sample into a cold trap or collecting the volatile materials on an absorption medium followed by extraction using, e.g., heat or a liquid or gas. Silicon, activated carbon, etc. can be used to collect volatile materials for subsequent release to permit testing. Absorption or concentration techniques are especially desirable for animals and people that cannot be asked to continue to breathe into a tube to permit collection of sufficient volatiles for testing. The animal can be confined in an enclosure and the air in the enclosure continually exhausted into, e.g., a cold trap, an absorption media, etc.

In order to base health information on data obtained as discussed herein, e.g., from volatile materials, it is desirable to minimize or eliminate extraneous variables including mouthwashes, toothpastes, etc. Desirably, the effect of fasting and/or specific diets such as vegetarian, high protein, high carbohydrates, etc. is determined by comparing data obtained on a group of individuals who either fast for 24 hours or eat a specific diet, the data being obtained continuously, e.g., daily, until the data stabilizes. Absence of significant differences in the results provides confidence that the selected variables do not affect the test results. The stable data can then be used for comparison to detect changes. The selection of the kind of diet that is best for establishing a standard, the length of time the diet must be followed to reach a stable condition and the timing for testing is desirably selected for each individual based on similar tests for similar individuals with similar backgrounds and diets. Thus, the databases used herein are desirably based on data collected from individuals using conditions determined at least in part by the results of this research.

The diet of the entity being tested is desirably controlled, or limited, or noted before and/or during the test to avoid, or compensate for, variation due to diet, and especially if one is testing the effect of diet on the condition of the entity.

For determining non-food effects, it is often desirable to limit the intake of food. I.e., it can be desirable to collect a “fasting” sample.

Concentration techniques are useful in the present invention so long as the same techniques are used for developing the reference sets and analyzing the specific entity. However, it should be noted that if the concentration technique changes the pattern, the results should be compared to results obtained using the same method of concentration. Concentration is less needed with the method of this invention when patterns are used as a basis for comparison as opposed to using the absolute amount of some given compound to determine the condition of an entity. Concentration is desirable when preparing reference sets as disclosed herein to ensure that sufficient amounts of materials present in very small quantities are collected.

A method for sample collection herein can also comprise “staged” collection and testing wherein the results of an initial test such as a test on the total volatiles emitted by an entity when breathing are analyzed and the analysis used to select a subsequent test involving a different sample collection such as a test on the volatiles from the urine, feces, sweat, feet, etc., which are the most likely source to provide results that will improve the diagnosis. Similarly, the initial test can be used to select other types of follow-up tests such as tests using a different multivariate analytical test or a comparison of the test results with different reference set test results relating to specific diseases or conditions. A method herein can also comprise selection of the test conditions (E.g., diet, temperature, weight loss or gain, presence of a rash, environmental exposure, common diseases found in the area at the time of testing, etc.) based upon observation of the individual or knowledge of the local situation.

In methods for doing research on the health, aging, and/or diseases, of living entities, it is desirable to have as much information as possible. The use of one, or more multivariate analytical techniques to study samples, e.g., vapor phase chromatographic analysis of volatile samples, derived from the breath, the skin, the urine, the feces, etc., of living entities of different known populations, e.g., young healthy, older healthy, young and/or old unhealthy entities with various known diseases or inherited differences, either alone, or in combination with one or more other multivariate analytical techniques, followed by comparison of the results from these populations using “pattern recognition” or other software designed to identify which variables in the sample populations are most different from the same variables in a normal population, can allow one to identify the “markers” that distinguish the populations and/or their aging and/or their conditions.

The above collection of samples from e.g., humans, animals, etc., can be combined with the taking of other information such as blood pressure, weight, temperature, digital pictures of the individuals and/or reference pictures representing the appearance of conditions like rashes and other skin conditions to provide permanent records of the individual and to allow specialists to make diagnoses from remote locations. The methods can also use digitized representations of the entity's structure with indications from, e.g., the entity or separate analytical techniques, to clearly make of record the location of a pain, a swelling, etc., for incorporation into a data base for searching to determine patterns.

Collection of data from homogeneous populations having similar genetic backgrounds and similar diets is especially effective. Such populations occur in many African countries where the population is mostly from one or two tribes and the diet is dictated by subsistence conditions.

C. Reference Sets

Patterns provide much more information than individual “markers”, since individual markers can be associated with more than one condition. However, use of patterns requires the creation of reference sets on known populations to obtain the characteristic patterns associated with those populations.

The material, especially the volatile material, that is analyzed herein is desirably collected from at least one of the breath, urine, feces, saliva, or surface covering (e.g. skin) of a living entity, either under ambient conditions or under conditions that increase the amount of volatile material collected, e.g., heat, vacuum, treatment with one or more media, etc. As more different types of samples are collected from more sources, there will be more information and therefore more indication of the status of the living entity. Desirably, the results are obtained quickly and it is also desirable, for completeness, to obtain results from more than one source from an individual, e.g., from the breath, urine, feces, etc. and collecting, e.g., volatile materials in a way to maximize collection, e.g., in a “cold trap” or by absorption on a media for subsequent release, e.g., by heating, displacement by a solvent such as water, etc.

To create a reference set, samples are typically collected at more than one time, either from the same living entity to create an individual's own reference set, or from several similar living entities to create a reference set for the similar population of entities, under conditions that are controlled to minimize variations due to condition of the environment as opposed to variations due to the condition of the entity or entities. Multiple samples increase the accuracy of the method since the effects of any variables that are not controlled is thereby minimized. The need to have a statistically significant difference, or similarity, determines the number of samples taken, the method of collection, the number of sources for samples used, etc. Statistical significance is desirably high, but embodiments reflecting increasing significance of e.g., 60%, 70%, 80%, 90%, 95%, and 99% provide increased confidence in the value of the results.

Each sample of the volatile material is desirably collected and analyzed using the same analytical apparatus and standard methodology. Thus, in one aspect of the invention, there is a central database or “reference set” database and the addition of data to the database(s) is restricted to data meeting certain criteria, in order to avoid data that contains misleading and/or inconsistent information and to maximize the accuracy. In order to minimize human error, the analysis is typically performed automatically by machine to minimize the uncontrolled variables due to human error. It is desirable to run each analysis using a computer and store the results of the analyses in machine-readable, e.g., digital, form. For convenience, it is desirable to have the control program, the data analysis, and the results of the analyses all stored in digital form in a standard format. The central or reference set database is desirably one that can be accessed, at least in part, by health care professionals, especially those who agree to keep the information confidential. Public health officials can be considered health care professionals for the purpose of accessing the database.

The results of the analyses are desirably stored in a database with indexing by a variety of variables, e.g. age, sex, location, diagnoses, etc., to permit retrieval of analyses from entities that share one or more similarities. Desirably the results are stored with additional information about each of the living entities which includes at least one of the identity of the specific entity, the age, sex, race, weight, diet, national origin, medical history, observation of condition, or species of the specific living entity.

For creation of reference sets, the results from repetitions of tests on a single individual are initially treated as an individual class and reference set. If the interclass difference between a first individual's results based on multiple tests (the first individual's class) and the results from other individuals' variations in results, the second, third, etc., individual(s) classes, are closer than the intra-class differences within the individual classes, the classes can be combined to create a new combined class reference set representing all of the individuals. Desirably, the classes/sets for several individuals, e.g., about 10 or more, individuals sharing the same basic characteristic variables, are combined to create a reference set representing all individuals sharing the same basic characteristic variables. The number of individuals needed to establish a class can be determined when establishing the “normal” class. There is a need for each individual to have that individual's baseline established. Therefore, there is no needless duplication at this time. Once a reasonable number of individuals have been tested, analysis of the data will determine the closeness of the data and the degree of variation within the group. Subgroups can be selected and the effect of different variables studied for the purpose of establishing appropriate subgroups for standards and for selection of subsequent individuals who have an abnormal condition to compare with the selected subgroup.

When the results from two such classes/sets representing groups of individuals that do not share all of the same basic characteristic variables are sufficiently similar, they can also be combined into a reference set representing both groups. When classes of individuals are different, i.e., the inter-class difference between classes is significantly different than the intra-class difference within the classes/sets, the two classes/sets will normally require the use of separate reference sets.

The above process can then be repeated using individuals having both the same basic characteristic variables and a common disease or abnormal condition to obtain data to create reference sets for each disease/condition. Desirably, the reference sets for normal individuals, and the reference sets for those individuals having a given disease or abnormal condition, are based upon a population of individuals having the same basic, characteristic variables. Separate reference sets are created for each disease or abnormal condition. However, such reference sets from populations having the same basic characteristic variables can be combined into a more generic reference sets based on individuals having different basic characteristic variables so long as the interclass differences are smaller than the intra-class variations. Similarly, combined reference sets representing more than one disease can be created. In this situation, subsequent work to make the diagnosis will be required. In each instance the combined reference sets can be used when the inter-class distance from the combined normal reference set to a combined reference set representing one or more abnormal disease/condition(s) is less than the intra-class variances within the combined normal reference set and the abnormal disease/condition reference set. If reference sets representing different diseases or abnormal conditions are too close to determine which disease or abnormal condition is present, the final diagnosis will depend upon additional tests and observations.

Reference sets representing normal healthy individuals and one or more diseases or abnormal conditions are selected to be a reference set. The next step is to use one of the usual clustering methods such as Hierarchical Cluster Analysis (HCA), factor analysis techniques such as Principal Components Analysis (PCA), or any other statistical or discriminant analysis test, to verify that each of the selected reference sets do possess the requisite inter-class differences that are statistically significantly larger than the intra-class variances within the selected reference sets. Those skilled in the art will recognize that significant difference can be defined by a variety of statistical criteria.

A classification model is built for the reference set using one of the classification methods available such as the SIMCA classification (Pirouette v4.0, Infometrix, Inc.) to determine the inter-class distances between the reference sets.

D. Methodology

A desirable method for practice of the present invention comprises creating a database comprising representative reference sets for diagnostic purposes. The first step comprises creating one or more digitally defined pattern reference sets representing normal healthy individuals and other reference sets representing individuals having one or more diseases or abnormal conditions as discussed above. The reference sets are desirably derived from individuals representing the different basic, characteristic variables most likely to affect the results. Specific selections of basic, characteristic variables can include different sexes, different diets [normal for different nationalities, vegetarian (or vegan), fasting, etc.], different races, etc. The number of different individuals needed to represent each basic, characteristic variable depends upon the reproducibility of the tests on the individuals and the results of preprocessing as described herein but desirably 6, 10, 20, or more individuals are used to improve the accuracy of the reference sets. The results for any individual can also be used as a reference set for that individual to compare to results of subsequent tests to detect any changes in the condition of that individual as discussed herein.

The source of the samples for the reference sets is conveniently the breath of each individual. However, if the samples from the breath do not provide a reference set for a specific disease or abnormal condition that is significantly different from the reference set for individuals having a normal health condition, it is desirable to create reference sets collected from over urine, feces, skin at specific locations such as armpits or feet, etc. Also, if the method of collection of the volatile materials from the breath or over urine, etc., by simply collecting a volume of air does not provide a satisfactory separation of the normal health reference set and the disease/abnormal reference sets, the air can be concentrated as discussed hereinbefore. The reference sets are desirably created for use together using as close to identical methods as possible. The reference sets should have inter-reference-set differences larger than the intra-reference-set variances.

A desirable method comprises the following steps: (a) select a number n of distinct reference sets comprising health conditions of interest, and which are used to define an n-dimension space; (b) using headspace mass spectrometry, determine the mass spectral channel signal pattern of the headspace derived from an individual multiple times, and use the combined results to define a point representing the individual; (c) define the location of said point in said n-dimension space, by determining the differences between said point and each of the reference sets, by defining the distances from said point to each of the reference sets.

In another desirable method, a number of reference set samples are chosen that are representative of, and span the range of conditions likely to be of interest for determining the condition of a specific entity to comprise the basis for a number of reference sets; multiple measurements are made on each reference set sample; the data for samples representing each reference set for a specific condition are optionally pre-processed by vector normalization; optionally, but desirably, verify that differences within each reference set are smaller than differences between it and each of the different reference sets representing different conditions; optionally, build a classification model for the reference sets; make multiple measurements on samples derived from the specific entity; determine the “distance” from the test samples of the specific entity to the reference sets; and optionally display the result to visually depict the similarity or difference of the said specific complex material relative to the reference sets.

A desirable specific method for objective classification of volatile materials given off by living entities can comprise the following steps:

1. One or more reference samples of volatile materials are collected from living entities, desirably sharing characteristics relevant to the composition of the volatile materials, and sharing a known condition status, e.g., good health or having a common health problem. Especially at least about 5, more desirably at least about 10, and even more, reference samples are used to create a reference set. Multiple reference sets can be created that are representative of and span the ranges of conditions, or status, that are likely to be encountered for the specific entity. Typical descriptors for the sets are the names of diseases and/or conditions as well as the identifying information for the characteristics of the entity or entities used for collecting the data.

2. Make multiple (n) measurements on each reference sample. Measurements are typically done by headspace-mass spectrometry. Data obtained are the mass spectral channel signals (m/z) after electron-ionization of the volatiles in the headspace over the sample. More generally, data can consist of signals from any multivariate analysis system or be a collection of univariate data obtained from several independent measurement techniques. Replications of the (n) measurements, desirably at least about 3 times, more desirably at least about 5 times, should be made on each reference sample. Each set of replicates for the samples from an entity or entities having a specific condition or status is hereinafter referred to as a reference “class”. Where it has been demonstrated that the differences in the characteristics of the entities do not affect the data, samples from several entities can be combined for convenience in processing.

3. Pre-process the data. The data in this example are pre-processed by vector normalization; however there are several options for pre-processing depending on the type of data obtained.

4. Verify distinctiveness for the reference sets/class. Verify that differences within a reference set (class) (intra-class variance) are smaller than differences between reference sets (classes) (inter-class variance). This can be done using any of a variety of clustering methods, such as Hierarchical Cluster Analysis (HCA), factor analysis techniques such as Principal Components Analysis (PCA), or any other statistical or discriminant analysis tests. Those skilled in the art will recognize that significant difference can be defined by any of several statistical criteria.

5. Build a classification model for the reference sets. Apply SIMCA classification (Infometrix, Inc.) to the reference sets to determine the inter-class distances between the reference sets/classes.

6. Make multiple (n) measurements on test, or unknown, samples obtained from specific entities. Test samples can be unknown samples or any samples for which objective classification relative to the reference sets is desired. Replications of the (n) measurements, desirably at least about 3 or more times, more desirably at least about 5 or more times, can be made on each test sample in an identical manner as for the reference samples.

7. Determine the “distance” from each test sample to the reference sets. Here, test samples are compared to a set of reference samples for which the entity and/or its status is known and well-defined. Ideally, the reference samples are based upon multiple entities or populations. In typical use, the SIMCA classification model constructed from the reference sets/classes would be used to “predict” the class assignment of test samples. This determination is based on how well the test sample can be modeled by any one of reference class models, relative to the others. A good or poor “fit” into a class depends on the magnitude of a test “residual” or error of fit. In this example, an average “distance” is calculated from each test sample to each of the reference classes. A smaller distance implies similarity to the reference class, while a larger distance implies difference.

8. Optionally, but desirably, display results that visually depict the similarities or differences of the test sample(s) relative to reference sets or samples. This can be done using a radial plot (also called “spider diagram” or “star plot”) in which each of the reference classes is a terminal set. The “distance” of the test sample to each of the reference classes is plotted graphically. In situations where the reference samples are readily characterized by descriptors and the measurements can be shown to correlate with these descriptors, then the similarity or difference of the test sample relative to the reference can be used to predict the condition or status of the entity that provides the test sample as compared to each of the reference conditions or status.

Tests are run on an individual (repetitions as required to achieve consistency) for whom a diagnosis is desired using the same methodology used to create the reference sets. The distance between the results for the individual and each of the reference sets is determined. The closeness of the point representing the individual to a reference set representing an abnormality/disease is a measure of the likelihood of the individual having the disease/abnormality represented by the reference set.

By using suitable reference populations of entities, each population having a known status, to create reference sets (sets), it is possible to classify the status of the test entities by their distance from these references, i.e., as coordinates in a multidimensional space. Thus, a volatile sample obtained from an entity can be analyzed by vapor phase mass spectrometry and the results used to define the status of the entity, at least in part, independent of other information. Such a method can determine the existence or absence of multiple abnormalities using a single non-invasive procedure that takes only a few minutes and which can be duplicated readily as situations change to provide a history of those changes.

The number of reference sets for comparison can be selected to represent only the most likely conditions that might be found in a given population or based upon observation of the test entity to minimize data transmission and/or analysis.

Desirably the methods herein utilize a computer-readable medium containing instructions for comparing an analysis of a target specific sample, said analysis being derived from mass spectrometry, nuclear magnetic resonance spectroscopy, gas chromatography, infrared spectrometry, and/or Raman spectrometry, electronic nose, image analysis, or combinations thereof, to more than one reference set where said reference set is based on a similar analysis of other standard complex materials and defining the target complex material represented by said target specific sample in terms of the differences between the target complex material(s) and the reference set, or sets, representing the standard complex materials(s).

E. Health Care Methods Based on Single Entity Reference Sets

In the simplest case, data is collected on a single living entity, desirably for multiple times over a period of time, to minimize the effect of uncontrolled variables and desirably when the entity is “normal”. Variables that can be controlled, like diet, time of day, method of collection, method of analysis, apparatus, computer programs, etc. are typically controlled as much as possible and documented to allow for the subsequent correlation and/or analyses of their affect on the results and reproducing the test conditions for subsequent tests. When the data is consistent, the results can be considered a “reference set” for that specific living entity. Subsequent tests are compared against this reference set and any significant difference is evidence of a change in the condition of the living entity. Such changes can signal either the existence of a problem or an improvement in condition. If there are no changes, one can use the information as an ongoing confirmation and/or improvement of the reference set's accuracy.

The methods herein are especially useful for detecting and/or diagnosing changes in the condition of, a living entity. Such methods herein can be used to detect conditions like cancer, impending heart attacks, impending strokes, etc., and such methods can also be used to track the effects of diet, specific treatments, etc. These methods are also especially useful for humans who cannot provide information and animals.

Desirably, the invention uses multivariate analytical methods where the volatile materials are collected in a non-invasive way. The non-invasive nature of a test makes it easier to obtain multiple samples without resistance from, or injury to, the entity. The source of the volatile material can optionally be selected for optimum value from limited tests when the specific risk is known. For example, one could collect the sample from urine when bladder cancer is suspected and from feces when colon cancer is suspected.

The methods can help determine the relative levels of materials created by an entity's natural life processes. The changes in the materials given off by an entity provide valuable information about its condition. The more variables that are measured, the more information can be obtained. If no particular condition is suspected, the tests are desirably conducted on all of the various sources of volatile materials and using more than one multivariate analytical technique. Therefore, it is highly desirable in the practice of the invention to test materials from more than one source including the breath, the feces, the urine, the saliva, and the skin (e.g., sweat or decomposition products), especially when there is no specific condition suspected and especially when the individual's reference sets are being created. As a goal, it is desirable that the database should contain information from all sources under a variety of conditions that reflects all situations of interest.

Mammals normally get rid of the waste products from the chemical processes used by their bodies, e.g., to produce energy, by expelling them in, e.g., the breath, sweat, urine, and/or feces. These complex processes in, e.g., mammals, result in varying amounts and/or ratios of different volatile materials based on many factors that are not normally controlled, but some of which can be controlled. The present invention uses multivariable analytical methods like vapor phase spectrometry to determine the amounts and/or ratios of many of the materials excreted by entities. While this information can be useful, it is much more useful when compared to e.g., historical information from the same entity to determine whether there has been a change, and what the change is. Also, as discussed hereinafter, the condition of such living entity can also be compared to the results of such multivariate analyses on a population of similar entities, typically where the analyses are carried out under similar conditions.

This method is especially useful for providing health care to a living entity. The method is desirably used in combination with other information such as weight, height, temperature, blood pressure, heart rate, etc., that are part of a normal physical evaluation. I.e., the information from a multivariate analysis can be used either alone, or, especially, integrated with other information. The information obtained from these multivariate analyses can be used alone or used by a trained observer along with information obtained by, e.g., a general test protocol and/or observation. Typically, health care professionals are the trained observers. General test protocols in routine annual health checks include taking the weight, the height, the blood pressure, the pulse rate, etc. The results of such multivariable testing can be used by, e.g., a health care giver, as part of the basic collection of information used to help determine the health status of patients.

When such testing has been done over a period of time on a specific entity, any deviation in results is especially valuable to help determine if there is any health change. The pattern that results from a complex multivariable analysis of, e.g., the volatiles given off by an entity provides a much better picture of the overall health than the analysis for any specific volatile “marker”. Specific markers, by their nature, help confirm the existence or extent of a specific problem that is already suspected. The tests on multiple variables can help determine the existence of an unknown problem, or an early warning of a change either in an existing problem or the start of a new problem or side effect, and with the proper reference sets suggest the cause, leading to, e.g., more specific tests, a more effective diagnosis, and/or follow-up treatment.

The testing can be used to provide feedback to human patients and the health care provider. The speed with which the results are obtained allows for modifying treatments to obtain better results. In life threatening situations, the shorter time that elapses before the results are known on the effectiveness of a treatment, the more likely it is that a good result will be obtained. Also, when the patient sees the beneficial results of taking a medicine, behavior modification, diet, etc., there is a much greater likelihood that the patient will continue the helpful action. The method can also be used for “biofeedback”, with only a short time elapsing between sampling and results.

These tests can supplement, or supplant, the use of trained animals. Thus, in one aspect, the method is used on a regular basis to test individuals known to have potential life threatening conditions such as heart conditions. The samples can be taken by the individual that provides the samples and the initial analysis can be done at, e.g., home and the data transferred electronically to a central location for comparison with the individual's history, other data, etc. The need for a trained animal as a constant companion can be evaluated based on facts to ensure that such valuable animals are used to the greatest effect.

There is currently a great deal of effort to provide means for collecting health information on individuals automatically and transferring the information electronically to another site where it can be examined, evaluated, and/or saved. The present diagnostic procedure can provide information on a large variety of possible health conditions, including conditions that either have not been previously diagnosed, or which appear later. The present diagnostic procedure can monitor the progress of existing conditions without having a trained professional in constant attendance.

F. Health Care Methods Based on Multiple Reference Sets

In order to obtain information in an efficient manner, specific data from a specific individual is desirably compared first with the individual's own history to determine any changes. If there is a change, the second comparison is desirably made with one or more reference sets based upon: (1) the individual's history and/or genetic disposition if there are no other logical indications; (2) if there is an indication of an infection, such as an elevated temperature, rash, etc., the comparison is made between the individual and reference sets based upon the most common diseases currently found in the same geographical area or in areas recently visited by the individual; (3) any other reference sets suggested by the health care professional caring for the individual, e.g., based upon observation of the individual. Any results that are not similar to any of the original reference sets should be compared to all of the known reference sets and if no match is found, public health authorities should be notified.

The analysis of samples, e.g., using vapor phase mass spectrometry can be accomplished with commercially available equipment in minutes, yet the results of such analyses can successfully predict the status of complex entities, especially when multiple complex reference sets are used.

The multiple reference sets from populations of similar individuals desirably represent representative conditions including: good health; common diseases; different ages; inherited deficiencies; etc. The present method can be used for diagnosis of disease, establishing the degree of health, etc., even in complex entities like pets, humans, etc. as well as plants.

The present method differs from existing methods such as those used to confirm the existence of a specific suspected condition, in that the present method can be used before the identification of specific markers by using complex reference sets derived from populations with the same characteristics to make the initial identification of a condition easier. Analyzing the different populations, one can utilize different sources of the volatile materials, different concentration techniques, etc., until a significant difference is found. The significant difference provides assurance that the test will identify the existence of, e.g., a specific disease, abnormality, etc. Additionally, the patterns can be used to help identify the specific marker, or markers, that are associated with the diseases. Computer software can identify which of the many variables is or are responsible for the significant difference. This opens the possibility of determining not only the marker that identifies the disease, but also other factors that are associated with the disease so that dietary causes, genetic causes, etc., can be identified.

Although the detection can be made more precise by using multiple reference sets representing, e.g., illnesses and/or conditions caused by known dangerous materials and/or their breakdown products, so long as the reference sets include the “normal” results, the method can detect abnormal situations. Just knowing that there is something different can alert one to check more thoroughly.

G. Digital Expression of the Data

Since the comparison of so much information is time consuming, it is highly desirable that the test method is able to provide the results in machine-readable form, especially digital form. It is also desirable that the operation of the analytical testing apparatus be done using a computer program in machine-readable form to avoid human variables. It is also desirable that the comparison of results be done using a computer and pattern recognition software. It is even more desirable that the results of tests on the same entity and the results of tests on populations of similar entities (reference sets) and on many entities other than the test entity in data bases for subsequent comparison. It is even more desirable that the information be kept in one or more data bases with other information relating to each entity such as age, health, sex, race, life style, diets, etc. to enable comparison of similar individuals having similar histories to simplify and speed comparisons.

These databases desirably are protected against unapproved access and all who have access desirably should agree to confidentiality requirements. This is an important safeguard against unapproved access and duplication of portions of the database. These databases and/or the computer programs or portions thereof are desirably copyrighted to provide a legal basis for protecting the information in the databases.

The storage of the results in digital format allows one to keep and retrieve data for comparison. In general, it is desirable to have a central database connected to individual health care providers, patients, etc. to improve the speed for making comparisons. It also provides a basis for detecting outbreaks of disease and/or health problems related to common factors. Thus, the promotion of public health using the methods herein is a special benefit.

For speed, and to minimize delays at the main database, it can be desirable to maintain an individual's records in a database at the individual's home, or at the health care provider's database. The individuals' characteristics are desirably included in the data base so that diagnostic reference sets can be custom selected from data obtained from individuals sharing the same basic characteristic variable(s) when it is found that the basic characteristic variable(s) are important to the comparison to determine if a particular disease/abnormality exists.

Central databases can be established in several locations to minimize the traffic at each database. The creation of reference sets can be expensive. Therefore, it will be desirable to charge for each comparison with a reference set to pay for the continual upgrading of that reference set.

The use of multivariate analytical techniques and multiple reference sets representing many known conditions, including healthy conditions, provides the maximum information on the condition of the entity in the least amount of time. Health care professionals can add the information obtained by such techniques to their existing tests and their own observations to maximize the information available. Observed information, which can optionally be obtained automatically, such as temperature, blood pressure, blood sugar, presence of a rash, etc., can be obtained, added to the database, and used to select appropriate reference sets to reduce the number of reference sets needed.

It is highly desirable that the reference sets be based upon populations of entities having known conditions, desirably where said conditions have been confirmed by either observers or other recognized tests for the conditions.

Thus, the present invention provides a convenient way to determine whether a living entity is healthy and provide useful information as to the status of the entity's health including indications of the cause of any abnormality. The present invention can also be used as part of a research effort to determine the specific “markers” related to different conditions so that more precise diagnoses can be made and/or more specific tests can be devised. In a research program, tests that provide not only patterns, but also compound specific information can be desirable.

It is highly desirable to use pictorial representations of, e.g., the external or internal physical conditions of test entities for incorporation in databases. Since health professionals typically take such conditions into account for determining health conditions/problems, inclusion of such information in digital form can be an important part of a pattern defining a condition Additionally, the use of pictorial representations can help clarify what a patient or health professional observes when there is a language difference, when the observer and the diagnostician are physically remote, etc. Digital photographs can be taken in, e.g., the visual spectrum, X-rays, or infrared, etc. depending on what is being studied.

H. Graphical Expression of the Data

It is desirable to display the data obtained in the methods herein in graphical form, especially for use by relatively untrained persons and/or people having different native languages. The use of “spider” diagrams can relate the position of the results of a test on a specific individual to the reference sets for several potential health problems to suggest which problem, or problems, are most likely to be the cause of that individual's health problems.

One can graph the values for the different variables and display them for comparison, both within a group of similar tests and for comparison with a similarly graphed “standard”. The peaks and valleys which exist can be compared using such graphs, although generally this kind of comparison is done more easily by computer programs to determine which particular variables are most important in separating one condition from another.

I. Other Specific Uses

Health Care Practice

In a health care method, one of the methods disclosed herein, e.g., comprising the taking of volatile sample(s) from a patient, and the analyses of the volatile sample(s) by a multivariate analytical process can be used as part of the initial data collection by a health care professional, similar to, and desirably in addition to, taking the weight, temperature, blood pressure, etc. With appropriate history and/or reference sets, the health care professional, or other care provider, can improve the diagnoses of an individual's condition.

The methods disclosed herein can be highly desirable when the patient objects to the care giver touching the patient or viewing parts of the patient which are concealed, e.g., for religious reasons.

Screening Tests

Other specific examples of practical uses of this technique include the development of screening tests, and/or the use of the screening tests themselves, for people, animals, etc. There are many situations, in addition to the treatment of patients by medical professionals, where there is a need to determine if there is any indication that entities have been infected with, or are carrying, dangerous entities, or are contaminated with dangerous materials. These kinds of screening can be readily accomplished in a matter of minutes using, e.g., vapor phase mass spectrometry. Although it only takes minutes to run vapor phase mass spectrometry tests with commercially available equipment, it can successfully identify complex materials including those that result from the complex processes in living entities. As used herein, “living entities” includes people and other mammals; reptiles, amphibians, fish, etc.; and can even include plants and simpler entities like molds, bacteria, etc., for screening purposes. The test methods herein can also be used to detect poisons, dangerous microorganisms, explosives, etc.

Research to Find “Markers”

Another example of a specific use comprises using a multivariate test method and available pattern recognition software to compare volatile materials originating from entities with normal health status and health problems to help establish the component(s) that indicate the health status/problem. Thus, the present invention also comprises the method of doing research to discover what volatile ingredients and/or levels of such ingredients are associated with specific problems, e.g., diseases, which then can be used to create more specific methods of detection and/or monitoring. The method can also be used to assist researchers in determining causative factors for health problems by aiding in the recognition of changes involved in health problems, etc.

As indicated above, it is possible to detect which variable, or group of variables, is responsible for separating one standard representing a particular condition from a normal standard. Isolating such variables can help one to determine what materials and/or reactions are associated with that particular condition. An analysis limited to the specific variables can be helpful in following the change in the condition, e.g., upon treatment, lapse of time, etc.

### Research to Determine Effects of Food or Medicine on Entities

This method can be especially useful in research programs for studying the effect of a food or medicine on living entities. The results can be used both individually and combined. Any change in the patterns from the entities exposed to the food, medicine, etc., will alert the observer that something is changing, either improving or becoming worse, as a result of the exposure.

The existence of such a monitoring system will allow public health agencies like the FDA (United States Food and Drug Administration) to monitor treated patients for the existence, or development, of potential problems.

The use of comparative software can also help one to determine what compound structure or structures are associated with an identified physiological effect. The identification of groups that contribute to the effect can help guide research into the creation of other compounds that possess, or in the case of undesirable effects, do not possess, a given physiological effect.

As discussed before, if one tests, e.g., a drug that causes the appearance of a volatile material associated with a given abnormal condition, one would not have to wait until the abnormal condition caused irrevocable damage or killed the human or animal before stopping treatment since the present methods could not detect the onset of the changes associated with the abnormal condition. With the methods of this invention one could detect the start of an abnormal effect immediately, thus allowing one to either begin to determine the nature of the abnormal condition or stop the test. A similar approach can be used to monitor patients during a treatment to detect adverse affects before serious damage occurs and/or to monitor progress.

Public Health Uses

In another method of studying the health of a population of similar living entities the methods herein are desirably used under controlled conditions over a period of time. The trend line is often more helpful than the results for any specific test and the results for a population can be more useful than the results for any individual. The CDC “United States Communicable Disease Center” could use this method to detect the early stages of an epidemic even before the particular disease is identified.

Such a process can provide much information that can indicate not only normality or the presence of one or more “abnormalities”, but can also give an indication of the size of any the difference between the normal state and the abnormal state. Once the reference sets are developed, the rapid screening of large numbers of specific entities can be accomplished readily.

One can use the present method, at least in part, to diagnose health problems and/or to establish the existence of potential problems, including dietary problems and monitor changes, either normally occurring, or changes caused by specific factors. In situations like warfare, or in third world countries where medical professionals are in short supply, the methods can be used alone as diagnostic methods, especially when transmitted by electronic means to a professional for study.

For public health purposes, a change in a population's test results can indicate that some basic important modification has occurred. For a specific geographical area, a change could indicate environmental changes or changes in the bacteria in the area. At the start of a pandemic, the change in the test results from a patient as compared to the population and known diseases present in the population could indicate the advent of the situation, major mutations in the agent, etc., allowing initiation of remedial actions or studies to determine remedial actions. For public health purposes, it is desirable to have the results of tests on patients forwarded on a routine basis to a central repository.

In an increasingly interdependent society, there is a continuing need to make sure that illnesses, including those intentionally caused by dangerous materials, entities, etc., are controlled. Also, the identification at an early stage of disease, poison, inherited conditions, etc., allows one to provide positive steps for improvement, especially at an early stage where intervention is more likely to succeed. As the cost of health care rises and the need for health care professionals increases, the need for rapid screening and rapid diagnoses becomes more important. Also, for some situations, early detection can be critical to treatment.

These health care methods can be used to alert individuals and health care professionals to the existence of life threatening conditions. The methods are especially desirable for use in the veterinary field and in treatment of babies and children or other subjects who cannot readily provide information on their condition. The ease of collection and the speed of analysis and the ability to share the results broadly provide important benefits, especially in the public health area and in efforts in support of public health, such as testing to ensure that food animals do not have dangerous conditions. The speed of identification of potential health problems can allow the initiation of treatment at an early time when intervention is most effective.

The ability to quickly and cheaply screen people, animals, etc. for abnormal conditions and/or dangerous agents makes society much safer. The method can provide assistance to health care professionals in their diagnoses of patients.

As discussed before, the use of this type of testing and methodology is especially useful for animals, since they cannot provide information. Veterinarians can use these tests to determine the age, health, nutritional needs, etc., of an animal and then treat it accordingly. For example, the techniques herein can be used to determine abnormal conditions at an early stage and then track the effect of various treatments on said abnormal conditions. Thus, treatments can be started, and even evaluated for effect, long before the abnormal conditions would normally be identified.

Insect and Microorganism Research

In one method herein, sample organisms such as mold, bacteria, viruses, etc., and/or insects such as bedbugs, which give off volatile materials, can be tested and the results used as a “reference set” for comparison to detect their presence and/or their condition. The analysis of each of these samples results in a pattern of analytical results that can help identify the entity and/or its state of being Replications are continued until the desired degree of accuracy is obtained.

The air from over a surface such as skin or a hard surface such as a kitchen counter can be tested to help determine what organisms are present. The air over bedding can be tested to determine the presence of bedbugs. The air over a forest floor can be tested to determine the presence of, e.g., truffles.

Complex entities can also be studied by studying other organisms present on the entity, especially when those organisms have a symbiotic relationship with the entity or reflect some variable of the entity like skin pH. This approach can be valuable where the other organism is present on the surface of the target organism and other approaches do not provide the desired information. The presence of one or more specific organisms can be suggestive of the status of the entity where the status is required for the organism to be present. The presence or absence of such organisms can be identified by comparison with populations that either have the organism or do not have the organism when the organism itself gives off volatile materials like a fingerprint.

One can also use the present invention to help predict the character of volatile materials that will be collected from an entity when it is infected, e.g., predicting what volatile materials will be added to the normal volatile materials collected from a human when the human is infected with a specific virus or bacteria by determining the specific volatile materials given off by the organism itself or by comparing infected and non-infected populations. One can use the process to help determine whether a human is infected with a new organism, even when there is no population of humans that has been previously infected by that new organism so as to be the basis for a reference set. The existence of an observed “difference” from known microorganisms will suggest that the new organism exists.

Different complex entities can be categorized and/or identified, by using a spider graph, to containing different reference sets/classes that represent populations of entities sharing the same physiological makeup. If the reference sets are similar to the entities, they can be used to detect new entities and/or variations of entities. Such information can be used, e.g., to simplify testing of, e.g., drugs on pathological organisms, to see if they affect the organisms in any way, including the tendency to create mutants where the organisms give off volatile fingerprints. The presence of a mutant organism normally results in at least some difference in the volatile materials released.

Thus, the method can be used in research to determine whether a treatment of an organism results in perceptible changes that are either desirable or undesirable. This method, with appropriate qualification, can be used to screen compounds for their effect, or lack of effect, on an organism. Quantification of the volatile materials that result from an effect of a compound on an organism can be used to determine the relative effectiveness or safety for the compounds that cause the effect.

It is possible to use the method to help determine what structures in medicines are responsible for a given effect, thus allowing one to predict medicines/foods with desired effects or to simplify research in developing such new medicines/foods.

The large number of potential uses for the invention can be represented by the following specific examples.

### EXAMPLES

### Example 1

The breath from an individual cow from a population of healthy cows is repetitively tested, using vapor phase mass spectrometry as the multivariate analytical test method (six repetitions), and the results combined to create a reference set for the breath of normal cows. The breath from each cow, trapped in an enclosed container, is continuously evacuated through a cold trap or absorption media to concentrate the volatile materials in the air and the volatiles are then analyzed using a Gerstel ChemSensor 4440 Chemical Sensor system. The results are combined into a reference set, first testing the individual cows' repetitions to determine if they are consistent and then after combining each of the sets of repetitions, testing the sets to determine if they are closer than the variation within the sets.

Subsequently, individuals from a herd of cows are tested to determine whether they are healthy by determining if the variation in repetitions for the test individual is the same or greater than the distance of the combined repetitions from the reference set. If the distance from the combined results of the test individual to the reference set for normal cows is not significantly different from the variation in the individual's test results, the cow is considered to be healthy.

A reference set is created in the same way for a population having a specific illness such as “mad cow” disease. The reference sets for the healthy cows and the cows having the disease are compared to see if the distance between the two reference sets is greater than the variation in the results within the reference sets to see if the difference between the two populations is statistically significant.

If the breath of the cows provides reference sets that are distinguishable between the normal cows and the diseased cows the reference sets are acceptable. If the reference sets are not acceptable the procedure is repeated with air from another source. Once one determines which source of volatile compounds is required to create the proper reference set, e.g., breath, urine, feces, etc., so as to create a reference set for a disease that is significantly different from the healthy cow reference set, one can then quickly identify which animals, if any, are infected. The individual cows are tested repetitively and the results compared to the reference sets. The sick individual cows are separated and either treated or destroyed. The cows that do not exhibit the illness are observed until they can be declared no longer at risk of coming down with the illness.

### Example 2

The method of Example 1 is used for humans. Individual humans are tested repetitively and the results combined for normal humans and humans having the same known diseases into reference sets representing humans having good health and humans having a specific disease for each reference set.

In the case of the recent “SARS” outbreak, one tests a population of ill people having SARS to create a reference set. The testing of individuals who were initially infected would establish how their volatile materials differ from those of a normal healthy population so that subsequent screening of exposed individuals could quickly determine if they carried the infection, and equally important, whether they were free of the infection. For example, the air in an airplane can continuously be evacuated through a volatile material trap and then checked near the end of the trip to determine if there is any significant amount of volatile material indicating a health problem for any of the people on board the airplane. Alternatively, the breath from each person could be collected and analyzed.

### Example 3

The headspace over produce that is known to be free of microorganisms that cause disease is tested repetitively using the multivariate analytical method of the previous examples to provide a reference set for such produce. Using the same analytical method on the headspace over produce being transported will determine if any abnormal living material is present when the results are significantly different. The presence of volatile materials indicative of some living material, or the presence of any abnormal volatile materials, indicates a need to check the produce more carefully.

Once a reference set is established for produce that is “safe”, subsequent shipments are tested to determine if there is a significant difference. The test prevents unsafe produce being sold until the danger is removed. Unlike specific testing, the present method uses complex relevant reference sets that are compared quickly to determine if there is a problem, even without knowing what the problem is. With appropriate reference sets one can establish what the problem is with a high degree of probability.

Reference sets are prepared for produce carrying various harmful microorganisms and those reference sets can be used to provide an initial indication of what harmful organism is present.

Thus, in the aspect suggested by Examples 1-3, the method involves analyzing, using a multivariate analytical method, one, or more, samples of volatile materials given off by a specific living entity, or entities, and comparing the analytical results from the specific entity to one or more “reference sets” derived from similar analyses of representative populations, each of which shares a known condition, to determine the condition of the subject entity or entities by determining which reference set is closest to the results from the test entity. Testing of the air over a population of entities in an enclosed space can establish the presence, or absence, of an abnormality in even one of the entities, so long as an appropriate reference set is available.

### Example 4

Samples of the breath of individuals from healthy populations of humans, dogs, cats, cows, horses, etc. are analyzed using at least one multivariate analytical method to create reference sets for each different animal. Desirably, the reference sets are representative of the populations of interest. E.g., for humans, one creates reference sets where the variables are sex (male/female), age, race (breed), dietary habits, etc., if those variables are found to affect the test results. Subsequent testing of breath samples from specific individuals would be compared in the first instance to the appropriate reference set to determine if there is a significant difference. If a significant difference is detected, the basis for the difference is established, if necessary by using more conventional tests. If there is no difference, the testing is repeated at regular intervals to determine if the individual remains healthy as part of an ongoing health care regimen.

### Example 5

Breath samples from populations of humans, dogs, cats, cows, horses, etc. that share a specific abnormality such as a specific disease, are analyzed using the method of Example 4 to create a reference set for that disease. The results of subsequent tests of individuals are compared to the reference set for the disease, and desirably to a “normal” reference set, to determine if the individual has that disease and/or how far a disease has progressed.

### Example 6

The procedures of Examples 5 and 6 are repeated using volatile materials given off by either the urine or feces or skin of the same populations to create sets of reference sets for comparison with results from subsequent testing of specific individuals using the same source for the volatile materials.

### Example 7

Renal failure in cats is normally identified only after the condition has deteriorated to a point where the cat is sure to die, so that the effect of diets known to provide prolonged life cannot be studied at times before the condition is life threatening.

A reference set for a normal population of cats is created by the method of Example 4 using vapor phase mass spectrometry. The animals are confined in an enclosed space that is evacuated through a volatile material trap and the entrapped volatiles are subsequently tested in a Gerstel ChemSensor 4440 Chemical Sensor system.

Individuals from a population of cats with indications of renal failure or potential renal failure are tested repetitively using vapor phase mass spectrometry to create one or more reference sets. Each reference set is created using cats with essentially the same degree of renal failure as found by current accepted tests for renal failure. The reference sets are selected to represent increasing renal failure as compared to the healthy cat reference set.

The multivariate analytical method is used to determine the results for specific cats with varying degrees of renal failure. These specific results are compared to the reference sets to determine the degree of renal failure. These results, as established by comparing the specific results to the normal cat reference set and the reference sets representing different degrees of renal failure, are then compared to the results of a standard test for renal to establish the accuracy of the present method in determining the extent of renal damage.

The test of the present invention, which is non-invasive and therefore more readily used on cats, is used to determine the lowest level of renal impairment that can be found with the test, i.e., the least amount of damage that will yield results that are significantly different from the normal cat reference set. The test is then used for routine screening and appropriate measures like dietary changes instituted as soon as renal impairment is detected. The effectiveness of various treatments used for renal impairment is evaluated by comparing, over time, representative populations treated with the different treatments and determining the effect of the treatment on the progression of the renal impairment.

A similar method is used when experimental drugs or treatments are being studied to detect the first indications of unwanted side effects before serious damage occurs. The present invention allows one to monitor the condition and stop the treatment as soon as adverse indications are observed. The method is used to determine the effectiveness of various treatments.

### Example 8

The methods of Examples 1-2 and 4-6 are applied to individuals such as people, animals, etc. entering the country to determine if they are carrying an illness.

### Example 9

The methods of Examples 1-2 and 4-6 are applied to animals, fish, fowl, etc. destined for consumption to determine if they are safe for food.

### Example 10

The methods of Examples 1-2 and 4-7 are used to test the efficacy of drugs, food, etc. in the treatment of disease, dieting, etc., to determine efficacy and/or the presence, or absence of significant side effects.

### Example 11

A health care method comprises collecting volatile samples from the breath, urine, feces, and/or skin of an individual, concentrating the samples by collecting them in a cold trap using dry ice, analyzing them using a Gerstel ChemSensor 4440 Chemical Sensor system, and maintaining the analyses in a data base in digital form. The results from the individual are compared with previous results from the same individual and with reference sets based upon analyses from samples of representative populations having known diseases, health conditions, etc. using a computer and pattern recognition software, the data being in digital form and maintained in a data base available, e.g., either via internet connection or alternatively available locally.

Alternatively, a health care professional makes a determination of the individual's health using the data, its comparison with available reference sets, and the other information and observations obtained in the normal course of treatment. E.g., the method is carried out by a doctor on humans or by a veterinarian on dogs, cats, horses, cows, sheep, rabbits, birds, etc.

### Example 12

A database in digital form is established comprising the results of tests carried out as in Examples 1-11, and the various reference sets, said database being searchable using data obtained from tests on individuals to compare the individual test results with data in the database. The database additionally desirably comprises reference sets derived by combining results from populations having similar characteristics, which allows the comparison of the results from an individual entity with such reference sets to identify populations that are similar to the individual results for the purpose of suggesting what the condition is for the specific individual entity.

### Example 13

This Example illustrates a desirable method of the present invention comprising creating one or more digitally defined pattern reference sets representing either (1) healthy humans, optionally differentiated by sex, race, diet, etc., and/or (2) one or more reference sets for similarly differentiated individuals having a given disease or abnormal condition, and optionally using these reference sets for comparing a pattern obtained from one or more individuals with the reference sets for (1) and (2) to determine whether the individual is healthy or has one or more of the diseases or abnormal conditions. The individuals' characteristics are included in the database so that the reference sets can be custom prepared from data obtained from individuals sharing the same characteristics when it is found that the characteristic(s) are important to the comparison.

1. Data Collection. Measurements are made by headspace-mass spectrometry and require an equilibration of about 100 μL of volatiles obtained from either (1) the breath of an individual or the combined breaths of a group, e.g., about 100, healthy individuals or (2) the breath of an individual the combined breaths of a group, either about 100 or as many individuals as available, of individuals either having the same abnormal condition, or individuals sharing a common illness. The volatiles are optionally concentrated, optionally concentrated in cold traps, in a 10 mL headspace vial at about 50° C., followed by an injection of about 3 mL of headspace into an HP 5973 benchtop single quadrapole mass spectrometer. Data obtained are the mass spectral channel signals m/z=46 to 200 after electron-ionization of the volatiles in the headspace. Six replications are made on each reference sample. Each set of replicates for a given sample comprises a reference “class”. Desirably, the samples are separated into results from groups of individuals sharing similar sex, race, dietary habits, etc., or groups having statistically representative characteristics when the illness or condition has clearly different volatile components. The breath is collected, alternatively, from the nose, the mouth, the nose and mouth, etc. so long as the collection process is the same for all samples. Similarly, the headspace samples are obtained from the headspace over saliva, urine, feces, skin, etc. to provide analogous reference sets.

a Data Processing for the Reference Volatile Samples. This step is optional if the samples are obtained from the combined breaths of a statistically representative group of individuals previously determined to provide results equivalent to the combined results from the individuals. The data in this example obtained from individuals are pre-processed by vector normalization using an integrated chemometric/pattern recognition software package (e.g., Pirouette v.4.0, Infometrix, Inc.) loaded on a high-performance personal computer. This step is useful for combining the results of the analyses of groups of individuals having similar, but different, characteristics, but having either normal status or the same illness/abnormal condition to provide a single reference class.

b. Data Analysis for the Reference Volatile Samples of Step 2. Hierarchical Cluster Analysis (FICA) of the normalized data clusters the reference samples from individuals or groups having different characteristics into classes which are defined by (1) healthy individuals and (2) individuals having the same illness/abnormal condition by calculation of Euclidean distance. For example, the classes can comprise healthy individuals, individuals having kidney disease, individuals having measles, individuals having colds, individuals having diabetes, etc. The selection of the resulting dendrogram is acceptable if the intra-class distance (variation from, e.g., 6 replications) is smaller than the inter-class distance between the classes representing the various conditions. The dendrogram represents “Relative Health for Individuals”

This concept is further illustrated by the results of a Principal Components Analysis (PCA) which shows a 3-dimensional spatial relationship among the reference classes.

The samples obtained from groups of individuals having similar characteristics are used as reference classes when an individual with an unknown condition shares the characteristics of the group.

The reference classes are subjected to SIMCA classification analysis (Soft-Independent Modeling of Class Analogy) in order to verify class distinctions. An examination of interclass and intra-class residuals verifies that differences within a reference class are smaller than differences between reference classes. A matrix of interclass distances is constructed, where

$$Interclass distance , {D}_{12} = \sqrt{\frac{{s}_{12}^{2} + {s}_{21}^{2}}{{s}_{11}^{2} + {s}_{22}^{2}}} - 1 = {D}_{21}$$

The terms s₁₂ and s₂₁ are interclass residuals and s₁₁ and s₂₂ are intra-class residuals for classes 1 and 2, respectively. In this sense, D is essentially a ratio of standard deviations so a value greater that 3 is deemed significant at the 95% confidence level. Those skilled in the art will realize that the calculation of residuals, and hence interclass distances, depends on the number of factors retained for each class within the SIMCA model. As a general rule, the number of factors retained describes the majority of variance within a class, desirably greater than 90% but less than 100%. The number of factors chosen defines the reference set model and is consistent for all subsequent calculations.

2. Test Individuals. One or more replicate headspace-mass spectrometry measurements are made on volatiles collected from each of the test subjects. Measurements are made and data are normalized in an identical manner as the reference samples.

3. Test Individual Samples in the “Relative Health for Individuals Space”. All of the samples, test and reference samples, are projected into the SIMCA model created with the reference samples. A “distance” matrix is output that contains the sample residuals that result when attempting to project the test sample data into each the reference classes. This residual or “distance” is a measure of how well or poorly the sample is modeled by the principal features of each of the reference classes, and as such is a useful measure of similarity or dissimilarity. The closeness of an individual's results to those of the reference sets indicates the relative health of the individual and which, if any, of the reference conditions (e.g., illnesses) are present in the individual.

4. Test Individual Results in the Relative Health for Individuals Space. The display of results to visually depict the similarity or difference of the test sample relative to reference samples is conveniently accomplished with a radial plot (also called “spider diagram” or “star plot”) in which each of the reference sets is a terminal point. The axes are scaled by the “similarity index” S, a number calculated from the “distance” as follows, where d is the residual or “distance” value for any individual's test sample to a reference set (i) and d_{MAX} is the maximum distance value for the entire reference set:

Sᵢ=1−(dᵢ/d_{MAX})

wherein each Sᵢ has values from 0 (very different) to 1 (very similar). This way, the health of each individual can be defined digitally by the similarity indices S; relative to the reference samples (i), each of which is derived from individuals sharing the same illness. For each individual, the individual's similarity indices Sᵢ are compiled in the form of a “similarity matrix” using the class distances generated from SIMCA.

Failure of an individual's results to indicate either a healthy condition or one of the specific illnesses used for reference sets indicates the existence of another condition.

Subsequent testing in the same way, after treatment is initiated, is used to determine effectiveness of the treatment, onset of adverse effects, etc. A reference set is established using one or more collected samples from one or more individuals, or where the samples are shown to be similar for individuals, a combined sample obtained from more than one individual. Test samples prepared by combining materials selected from more than one individual can be used where the differences between reference sets are sufficiently large to detect a difference arising from only one of the individuals.

### Example 14

Example 13 is repeated with dogs and again with cats.

Failure of an individual dog's or cat's results to indicate either a healthy condition or one of the specific illnesses used for reference sets indicates the existence of another condition.

Subsequent testing in the same way after treatment is used to determine effectiveness of the treatment, onset of adverse effects, etc. Movement of the point representing the new test toward the normal reference set indicates improvement and movement toward the disease/abnormality reference set indicates continued worsening of the condition. Movement away from a line drawn between the normal reference set and the disease/abnormality reference set can indicate a side effect.

### Example 15

The method of Example 3 is used to identify soil, food, etc. that is the source of an infection. First, the air over a sample of the agent that is causing the infection is tested and used to create a reference set for that agent. Then the air over, e.g., soil, is tested and the results compared to the reference set to determine the presence of the volatiles associated with the agent. The contaminated soil can be treated until the virus, bacteria, fungus, etc. is no longer present as shown by testing.

### Example 16

The method of Example 13 is applied to a population that is homogeneous with respect to genetic background and diet with controls for age and sex.

What is claimed is:

1. A method for determining the condition of a living entity wherein at least one material given off from said living entity is analyzed with at least one multivariate analytical test method to provide test results and those results are then compared either to the test results from the same test method or methods on either the same entity at a time when the entity's condition is known, or to the results from the same test method or methods on one more reference individuals or reference populations of similar entities having a known condition.

2. The method of claim 1 which comprises a step in which at least one volatile material is collected at least once, in a standard way, from at least one individual living entity and analyzed using said multivariate analytical test method to provide test results that are used to create at least one reference set representative of the condition of said living entity.

3. The method of claim 1 which comprises at least one of:
(1) the step of developing one or more data bases, each of said data bases comprising one or more reference sets, and each of said reference sets comprising the test results from at least one multivariate analytical test method on materials given off by at least one individual from a population of individuals having similar or identical basic characteristics and a shared health condition, the reference set being used to represent all individuals having similar basic characteristics and the same said health condition;
(2) the step of using data mining software to establish a pattern in the test results in at least one of the reference sets of (1) where the pattern is representative of a population sharing a known condition;
(3) the step in which an individual is tested using the same multivariate analytical test method used to create the reference set of (1) and the individual's test results are compared to at least one of the said reference sets of (1);
(4) the step of (3) wherein said individual's test results are compared to the results from at least one reference set derived from at least one healthy population having similar genetic backgrounds, allergies, family histories of susceptibility to diseases, diets, and other relevant characteristics, to determine if the individual shares the same inherited basic characteristics as one of the populations;
(5) the step of using one of the methods herein as an initial step in the diagnosis of the condition of an individual;
(6) the step of using at least one pattern found by at least one of the methods herein to determine what body processes are related to a disease;
(7) the step of using at least one of the methods herein to monitor the progress of an individual's condition the step of using one of the methods herein to determine when changes occur in a microorganism or virus;
(8) the step of providing the data found using at least one of the methods herein in digital form for storage, retrieval, and/or analysis;
(9) the step in which one or more volatile materials are collected in a standard way, from at least one individual living entity and analyzed using said multivariate analytical test method; and
(10) the step of creating a reference set by analyzing volatile materials given off by at least one entity using at least one multivariate analytical test method, where all entities are similar to each other in at least one relevant characteristic, and combining the resulting test data into a reference set that is representative of results obtained by said multivariate analytical test method on other entities sharing the same relevant characteristic.

4. The method of claim 3 comprising at least one of:
(1) the step in which at least one database or reference set is copyrighted to provide a legal basis for protecting them from theft, and/or to provide a basis for receiving compensation for the use of the database or reference set to support the maintenance and improvement of said database or reference set; or
(2) the step of providing a standard food diet for test subjects to minimize differences resulting from diets.

5. The method of claim 1 in which the results of a test on a specific living entity are compared to at least one reference set representative of either the history of the entity itself or appropriate populations of similar entities sharing the same health condition.

6. The method of claim 1 comprising at least one of:
i. the step of using a computer and pattern recognition software for said computer to determine if one or more representative patterns exists within the results from a multivariate analytical test method on several individual entities sharing a similar condition;
ii. the step of using a computer and pattern recognition software for said computer to determine if one or more representative patterns found by (i) can be found in the test results of at least one individual entity;
iii. the step of providing a controlled diet for at least one individual living entity being tested using a multivariate analytical test method to minimize any effect on the results caused by the diet;
iv. the step of creating a health care system in which data from at least one multivariate analytical test method are collected at one or more times from one or more individual entities and kept in a data bank, optionally a central data bank;
v. the step of providing at least one representative picture in digitized form representing the appearance of at least one individual entity for use in diagnosis of said entity's health condition;
vi. the step of adding to a digital data base for an individual living entity at least one of the temperature and blood pressure taken at least one time;
vii. the step in which a health care professional uses at least one of (i)-(vi) in the care of a human patient;
viii. the step in which at least one of (i)-(vi) is used in the care of an animal;
ix. the step comprising analyzing the results from at least one multivariate analytical test method on a population of individuals having a common health condition and using data mining software to determine sub sets of variables that are most closely related to a said condition;
x. the step of using at least one graphical representation of the results of at least one test of using at least one multivariate analytical test method on an individual living entity having specific basic characteristics as compared to at least one reference set representative of, and comprising the results of, the same test method on a population of individual entities having the same basic characteristics;
xi. the step of testing a specific living entity using at least one multivariate analytical test method at a point near the specific entity, followed by pattern analysis and/or evaluation at another location;
xii. the step of determining the effectiveness of medicine, diet, or behavior changes by monitoring the effects using at least one of (i)-(xi);
xiii. the step of monitoring the health of a single individual living entity by comparing new results to historical results obtained by using at least one of (i)-(xii) on the same individual;
xiv. the step of improving the health of at least one individual living entity by providing feedback on positive and/or negative results obtained from changes in diet, medicines, behavior using at least one of (i)-(xiii); and
xv. the step of providing a basis for insurance risks for a living entity utilizing at least one of (i)-(xiv).

7. The method of claim 1 in which the entity is an animal.

8. A method for monitoring the health of many entities for public health purposes comprising the method of claim 1.

9. The method of creating a reference set using the method of claim 1 in which at least one volatile material is repetitively collected in a standard way from at least one living entity and analyzed using said multivariate analytical test method.

10. The method of claim 9 wherein said volatile material is collected from either the breath, urine, feces, saliva or surface covering of the living entity.

11. The method of claim 10 wherein said volatile material is concentrated.

12. The method of claim 9 wherein the results of the analyses are stored, in digitized form, in a data base.

13. The method of claim 12 wherein the results of said analyses are stored in a data base with information about the entity which includes at least one of: the identity of the specific entity, the entity's age, the entity's sex, the entity's race, the entity's weight, the entity's diet, the entity's national origin, the entity's medical history, the observed condition of the entity, and the species of the entity.

14. A method for determining the health condition of a living entity comprising at least one step selected from:
a, creating one or more reference sets using analyses of materials given off by representative entities sharing similar basic characteristics and the same health condition using at least one multivariate analytical test method according to claim 1 wherein the results of said analyses are representative of results from populations of entities sharing the same basic characteristics and health condition; and
b. analyzing at least one sample of volatile material similar to the materials used to create the reference set from a specific entity using the multivariate analytical test method to provide a specific test result; and comparing the specific test result with one or more of the reference sets.

15. The method of claim 14 wherein said entity is a mammal, the samples are obtained from at least one of the breath, urine, feces, and skin of the mammal and the comparison of the results is accomplished using the digital expression of the results, a computer, and pattern recognition software.

16. The method of claim 14 wherein said living entity is a mammal and the samples are obtained from the breath, urine, feces, and/or skin of the mammal and the samples are analyzed by vapor phase mass spectrometry.

17. A method for evaluating the effect of a medicine on entities comprising using the method of claim 1 on either a single entity or a population of similar living entities under controlled conditions over a period of time while the said entities use the food or medicine.

18. A method of providing health care to a living entity comprising the step of using the method of claim 1 and optionally providing the results to the patient, the health care provider, or both and selecting a treatment based upon the results.

19. The method of determining the effect of a diet for an animal species or humans on an abnormal condition comprising testing a representative population having the abnormal condition by the method of claim 1 while feeding the said diet to said population and continuing testing of the individuals of said population and combining the results to determine whether the diet affects the abnormal condition and, if so, how the diet affects the abnormal condition.

20. The method of studying animals using the method of claim 1 wherein the animal is confined in a box with ventilation and the air leaving the box is conveyed to a collection point for the volatiles given off by the animal.
